(19) United States
(12) Patent Application Publication
(10) Pub. No.: US 2024/0346834 A1
(43) Pub. Date: Oct. 17, 2024

(54) REFERENCE VALUE CREATING DEVICE, ALERTNESS LEVEL ESTIMATING DEVICE, AND REFERENCE VALUE CREATING METHOD

(71) Applicant: Mitsubishi Electric Corporation, Tokyo (JP)

(72) Inventor: Koki UENO, Tokyo (JP)

(73) Assignee: Mitsubishi Electric Corporation, Tokyo (JP)

(21) Appl. No.: 18/711,893

(22) PCT Filed: Dec. 13, 2021
(86) PCT No.: PCT/JP2021/045728

§ 371 Date: May 21, 2024

(51) Int. Cl. G06V 20/59; G06V 10/70; G06V 40/16

(52) U.S. Cl. CPC G06V 20/597; G06V 40/168; G06V 10/70

(57) ABSTRACT

A reference value creating device includes processing circuitry configured to; vehicle-related information related to a vehicle; determine a road type of a road on which the vehicle is traveling on a basis of the acquired vehicle-related information; acquire feature amount information regarding a feature amount of an occupant of the vehicle calculated on a basis of a captured image including a range in which at least a face of the occupant of the vehicle is present; and create a reference value of the feature amount for estimating an alertness level of the occupant on a basis of the acquired feature amount information, and create, when the determined road type has changed, the reference value of the feature amount corresponding to the changed road type.

### TECHNICAL FIELD

The present disclosure relates to a reference value creating device, an alertness level estimating device, and a reference value creating method for creating a reference value for estimating an alertness level of an occupant.

### BACKGROUND ART

Conventionally, there is a technique of calculating a feature amount indicating a state of an occupant such as a face direction of the occupant from information regarding the occupant such as a captured image obtained by capturing the occupant of a vehicle, and estimating an alertness level of the occupant in consideration of an individual difference by comparing the calculated feature amount with a reference value of the feature amount set for each occupant. Here, the tendency of a change in the feature amount varies depending on the type of the road (hereinafter referred to as “road type”) on which the vehicle is traveling. For example, when the feature amount is the face direction of the occupant, the face direction of the occupant is often changed in an ordinary road because stop at a signal or right/left turn at an intersection occurs more frequently than in an expressway. Accordingly, a technique of correcting the reference value of the feature amount according to the road type on which the vehicle is traveling is known. For example, Patent Literature 1 discloses a sleepiness calculator that corrects a sleepiness reference heart rate on the basis of the road type.

### CITATION LIST

### Patent Literature

Patent Literature 1: JP 2016-182242 A

### SUMMARY OF INVENTION

### Technical Problem

The tendency of the change in the feature amount depending on the road type also has an individual difference. However, the related art disclosed in Patent Literature 1 uniformly corrects the sleepiness reference heart rate with a preset value depending on the road type. As described above, in the related art, there is a problem that an individual difference in a tendency of a change in the feature amount depending on the road type cannot be considered in creating the reference value for estimating the alertness level of an occupant of a vehicle. As a result, in the related art, there is a possibility that the accuracy of estimation of the alertness level of the occupant is low.

The present disclosure has been made to solve the above-described problems, and an object thereof is to provide a reference value creating device capable of creating a reference value for estimating an alertness level of an occupant in consideration of an individual difference in a tendency of a change in a feature amount depending on a road type.

### Solution to Problem

A reference value creating device according to the present disclosure includes a vehicle-related information acquiring unit to acquire vehicle-related information related to a vehicle, a road determining unit to determine a road type of a road on which the vehicle is traveling on the basis of the vehicle-related information acquired by the vehicle-related information acquiring unit, a feature amount acquiring unit to acquire feature amount information regarding a feature amount of an occupant of the vehicle calculated on the basis of a captured image including a range in which at least a face of the occupant of the vehicle is present, and a reference value creating unit to create a reference value of the feature amount for estimating an alertness level of the occupant on the basis of the feature amount information acquired by the feature amount acquiring unit, and create, when the road type determined by the road determining unit has changed, the reference value of the feature amount corresponding to the changed road type on the basis of the feature amount information regarding the feature amount calculated on the basis of the captured image captured in the vehicle traveling on the road of the changed road type.

### Advantageous Effects of Invention

According to the present disclosure, it is possible to create a reference value for estimating an alertness level of an occupant in consideration of an individual difference in a tendency of a change in a feature amount depending on a road type.

### BRIEF DESCRIPTION OF DRAWINGS

FIG. 1 is a diagram illustrating a configuration example of a reference value creating device and an alertness level estimating device according to a first embodiment.

FIG. 2 is a diagram illustrating an example of content of reference value information stored in a storage unit by a reference value creating unit in the first embodiment.

FIG. 3 is a flowchart for explaining an operation of the reference value creating device according to the first embodiment.

FIG. 4 is a flowchart for explaining a specific operation of step ST4 in FIG. 3.

FIG. 5 is a flowchart for explaining an operation of the alertness level estimating device according to the first embodiment.

FIGS. 6A and 6B are diagrams illustrating an example of a hardware configuration of the reference value creating device and the alertness level estimating device according to the first embodiment.

FIG. 7 is a diagram illustrating a configuration example of a reference value creating device and an alertness level estimating device according to a second embodiment.

FIG. 8 is a flowchart for explaining an operation of the reference value creating device according to the second embodiment.

FIG. 9 is a flowchart for explaining a specific operation of step ST25 in FIG. 8.

### DESCRIPTION OF EMBODIMENTS

Hereinafter, embodiments of the present disclosure will be described in detail with reference to the drawings.

### First Embodiment

A reference value creating device according to a first embodiment creates a reference value of a feature amount for estimating an alertness level of an occupant of a vehicle. In the following first embodiment, the reference value of the feature amount for estimating the alertness level of the occupant of the vehicle is also simply referred to as a “reference value”.

The alertness level of the occupant is estimated on the basis of one or more feature amounts extracted on the basis of a captured image obtained by capturing at least the face of the occupant. Note that one or more feature amounts used for estimating the alertness level of the occupant are determined in advance. In the first embodiment, the feature amounts used to estimate the alertness level of the occupant are five different types of feature amounts (first feature amount, second feature amount, third feature amount, fourth feature amount, and fifth feature amount). The reference value creating device creates a reference value of each feature amount according to a road type of a road on which the vehicle is traveling, on the basis of the captured image and information related to the vehicle (hereinafter referred to as “vehicle-related information”). The reference value created by the reference value creating device is output to the alertness level estimating device, and the alertness level estimating device estimates the alertness level of the occupant on the basis of a reference value corresponding to the road type of the road on which the vehicle is traveling and the captured image. In the first embodiment, it is assumed that the occupant whose alertness level is to be estimated is, for example, a driver of a vehicle.

The alertness level of the occupant estimated by the alertness level estimating device is output to, for example, a driving assistance device, and the driving assistance device performs driving assistance such as warning to the occupant on the basis of the alertness level.

In the following first embodiment, the plurality of feature amounts, specifically, the above-described five feature amounts are also collectively referred to simply as the “feature amount”.

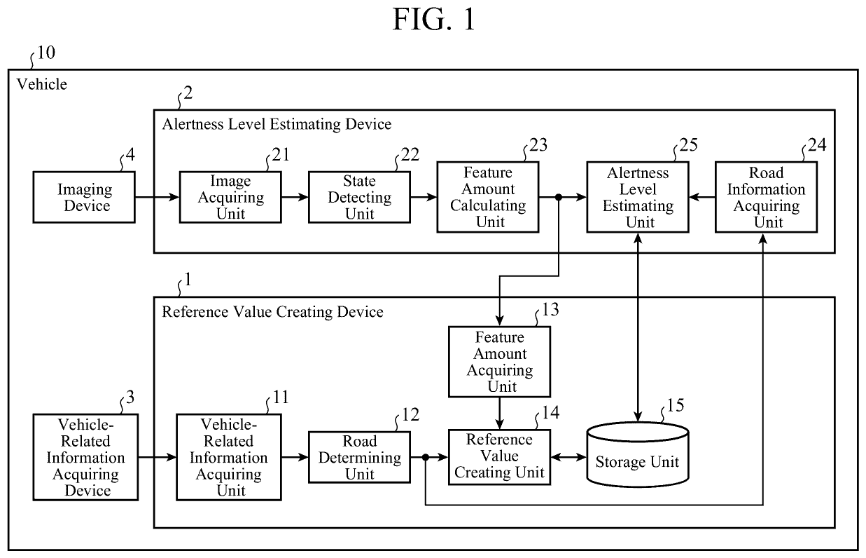

FIG. 1 is a diagram illustrating a configuration example of a reference value creating device 1 and an alertness level estimating device 2 according to the first embodiment.

The reference value creating device 1 is connected to the alertness level estimating device 2 and a vehicle-related information acquiring device 3.

The alertness level estimating device 2 is connected to the reference value creating device 1 and an imaging device 4.

The reference value creating device 1, the alertness level estimating device 2, the vehicle-related information acquiring device 3, and the imaging device 4 are mounted in a vehicle 10.

First, the vehicle-related information acquiring device 3 will be described.

The vehicle-related information acquiring device 3 acquires vehicle-related information. The vehicle-related information acquiring device 3 includes, for example, a vehicle speed sensor that detects a vehicle speed, a steering angle sensor that detects a steering wheel angle, an accelerator position sensor that detects an accelerator opening, a map database that stores map information, and a global positioning system (GPS). The vehicle-related information acquired by the vehicle-related information acquiring device 3 includes the vehicle speed of the vehicle 10, the steering wheel angle of the vehicle 10, the accelerator opening of the vehicle 10, the map information, and current position information of the vehicle 10.

The vehicle-related information acquiring device 3 outputs the vehicle-related information to the reference value creating device 1.

Next, the reference value creating device 1 will be described.

The reference value creating device 1 includes a vehicle-related information acquiring unit 11, a road determining unit 12, a feature amount acquiring unit 13, a reference value creating unit 14, and a storage unit 15.

The vehicle-related information acquiring unit 11 acquires the vehicle-related information output from the vehicle-related information acquiring device 3.

The vehicle-related information acquiring unit 11 outputs the acquired vehicle-related information to the road determining unit 12.

The road determining unit 12 determines the road type of the road on which the vehicle 10 is traveling on the basis of the vehicle-related information acquired by the vehicle-related information acquiring unit 11. The road type determined by the road determining unit 12 is defined in advance. In the first embodiment, as an example, there are two road types of “ordinary road” and “expressway”. In the first embodiment, the “expressway” also includes an automobile exclusive road. Note that this is merely an example, and for example, “narrow road” or “mountain road” may be defined as the road type. The road width of the “narrow road” is set in advance. In addition, “urban road” where there are many people or “suburban road” where there are few people may be defined as the road type. The road type can be a suitable type as long as it can be determined from the vehicle-related information acquired by the vehicle-related information acquiring device 3.

The road determining unit 12 determines the road type, here, “ordinary road” or “expressway” on the basis of, for example, the vehicle speed, the steering wheel angle, or the accelerator opening included in the vehicle-related information. The road determining unit 12 determines the road type on the basis of, for example, the vehicle speed, the steering wheel angle, or the accelerator opening, and conditions for determining the road type (hereinafter referred to as “road determination condition”). In the road determination condition, for example, conditions having the following contents are set. For example, when the following road determination condition is satisfied, the road determining unit 12 determines that the road on which the vehicle 10 is traveling is an “expressway”, and when the following road determination condition is not satisfied, the road on which the vehicle 10 is traveling is a “ordinary road”. Note that the road determining unit 12 just needs to store the acquired vehicle-related information for a predetermined period of, for example, one minute or more, and determine the rotation range of the steering wheel angle for the past one minute on the basis of the stored vehicle-related information.

### <Road Determination Condition>

An expressway is determined based on a range (hereinafter referred to as a “steering angle determination range”) in which a vehicle speed is equal to or more than a preset threshold (hereinafter referred to as a “vehicle speed determination threshold”) and the rotation range of the steering wheel angle for the past one minute is within a preset angle.

Note that, for example, in a case where “narrow road”, “mountain road”, “urban road”, or “suburban road” is defined as the road type, the road determining unit 12 just needs to determine the road type on the basis of, for example, the map information and the current position information of the vehicle 10 included in the vehicle-related information. It is assumed that the map information includes information regarding road widths of roads and information regarding whether it is an urban area or a suburb area.

The road determining unit 12 outputs information regarding the determined road type (hereinafter referred to as “road information”) to the reference value creating unit 14 and the alertness level estimating device 2.

The feature amount acquiring unit 13 acquires information (hereinafter referred to as “feature amount information”) regarding the feature amounts of the occupant from the alertness level estimating device 2.

The feature amount information is information in which the feature amounts of the occupant are associated with the captured image captured by the imaging device 4. The feature amounts of the occupant include, for example, an eyelid opening degree indicating an opening degree of an eye of the occupant, a mouth opening degree indicating an opening degree of the mouth of the occupant, a face direction of the occupant, a head position of the occupant, a line-of-sight direction of the occupant, a percent of the time eyelids are closed (PERCLOS) of the occupant, a movement of a mouth corner, the number of blinks of the occupant, or a blink speed of the occupant. These feature amounts are calculated in the alertness level estimating device 2 from the captured image captured by the imaging device 4. Details of the alertness level estimating device 2 and the imaging device 4 will be described later. Note that the alertness level estimating device 2 calculates the feature amount for each frame of the captured image. The feature amount acquiring unit 13 acquires feature amount information in which a feature amount is associated with each frame of the captured image in units of frames.

The feature amount acquiring unit 13 outputs the acquired feature amount information to the reference value creating unit 14.

The reference value creating unit 14 creates a reference value on the basis of the feature amount information acquired by the feature amount acquiring unit 13. The reference value creating unit 14 creates a reference value for each feature amount.

The reference value creating unit 14 creates the reference value when a preset condition (hereinafter referred to as a “reference value creation condition”) for creating the reference value is satisfied.

In the first embodiment, “when the power of the vehicle 10 is turned on and the vehicle 10 starts traveling (first condition)” and “when the road type of the road on which the vehicle 10 is traveling has changed, and a reference value of a feature amount for that road type is yet to be created (second condition)” are set as the reference value creation condition. The “first condition” is a condition for the reference value creating unit 14 to determine that it is time to create a reference value for the first time after the power of the vehicle 10 is turned on. The “second condition” is a condition for determining that it is a timing at which the reference value needs to be reviewed due to a change in the road type of the road on which the vehicle 10 is traveling, specifically, a timing at which a reference value corresponding to the changed road type should be created.

For example, the reference value creating unit 14 can determine, from the road information output from the road determining unit 12, that the road type of the road on which the vehicle 10 is traveling has changed. In addition, the reference value creating unit 14 can determine, for example, whether or not it is when the power of the vehicle 10 is turned on and the vehicle 10 starts traveling, or whether or not a reference value of a feature amount for a certain road type is yet to be created for the road type, from the information (hereinafter referred to as “reference value information”) regarding the reference value stored in the storage unit 15. The reference value information is information in which information that can specify a road type is associated with a reference value. When creating the reference value, the reference value creating unit 14 creates the reference value information and stores the reference value information in the storage unit 15. In the first embodiment, the reference value information stored in storage unit 15 is deleted, for example, when the power of vehicle 10 is turned off by a controller (not illustrated). For example, if the reference value information is not stored in the storage unit 15, the reference value creating unit 14 can determine that it is when the power of the vehicle 10 is turned on and the vehicle 10 starts traveling. In addition, for example, the reference value creating unit 14 can determine whether or not a reference value of a feature amount for a certain road type is yet to be created for the road type by referring to the reference value information stored in the storage unit 15. Details of creating the reference value and the reference value information by the reference value creating unit 14 will be described later.

When “the first condition” or “the second condition” is satisfied, the reference value creating unit 14 creates the reference value by determining that the reference value creation condition is satisfied. That is, the reference value creating unit 14 creates the reference value depending on the road type of the road on which the vehicle 10 is traveling. Specifically, when the power of the vehicle 10 is turned on and the vehicle 10 starts traveling, the reference value creating unit 14 first creates the reference value. Thereafter, for example, until the power of the vehicle 10 is turned off, the reference value creating unit 14 creates the reference value every time the road type of the road on which the vehicle 10 is traveling changes.

A specific reference value creating method by the reference value creating unit 14 will be described.

For example, the reference value creating unit 14 sets, as a reference value, a median of feature amounts included in feature amount information corresponding to captured images for a preset number of frames (hereinafter referred to as “the number of frames for reference value creation”) among the acquired feature amount information. For example, the reference value creating unit 14 may use, as the reference value, an average value of the feature amounts included in the feature amount information corresponding to the captured images for the number of frames for reference value creation, or may use a mode value of the feature amount as the reference value.

In addition, for example, the reference value creating unit 14 may use, as the reference value, a median, an average value, or a mode of the feature amount included in the feature amount information acquired in a preset period (hereinafter referred to as a “reference value creation target period”).

The reference value creating unit 14 stores the reference value information regarding the created reference value in the storage unit 15. The reference value creating unit 14 creates the reference value information by associating the created reference value with information that can specify the road type of the road on which the vehicle 10 is traveling, the information having been determined by the road determining unit 12, and stores the reference value information in the storage unit 15.

The storage unit 15 stores the reference value information.

Note that, in FIG. 1, the storage unit 15 is provided in the reference value creating device 1, but this is merely an example. The storage unit 15 may be provided outside the reference value creating device 1 at a place that can be referred to by the reference value creating device 1.

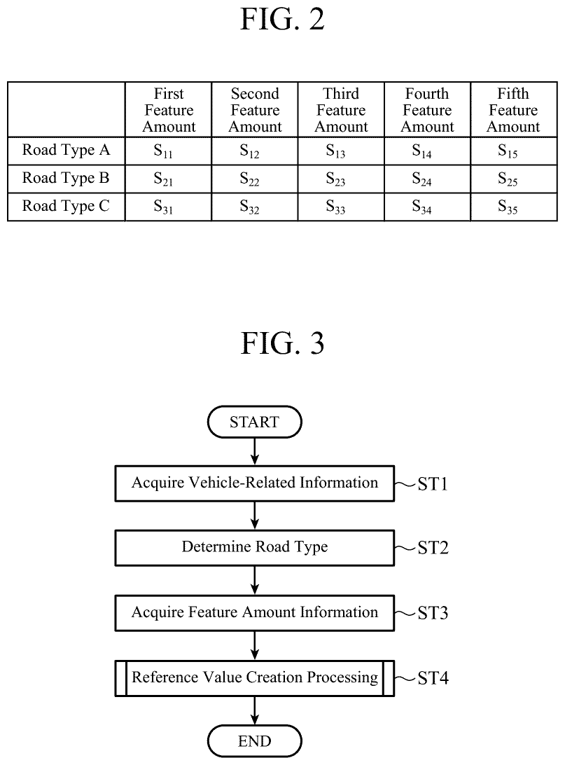

Here, FIG. 2 is a diagram illustrating an example of the content of the reference value information stored in the storage unit 15 by the reference value creating unit 14 in the first embodiment.

As illustrated in FIG. 2, the reference value information is information in which information that can specify a road type is associated with a reference value of each feature amount (first feature amount, second feature amount, third feature amount, fourth feature amount, and fifth feature amount).

FIG. 2 illustrates the reference value information in which the reference value of each feature amount is associated with each of three road types of a road type A, road type B, and road type C. For example, the reference value of the first feature amount is s₁₁, the reference value of the second feature amount is s₁₂, the reference value of the third feature amount is s₁₃, the reference value of the fourth feature amount is s₁₄, and the reference value of the fifth feature amount is sis corresponding to the road type A. The reference value of the first feature amount is s₂₁, the reference value of the second feature amount is s₂₂, the reference value of the third feature amount is s₂₃, the reference value of the fourth feature amount is s₂₄, and the reference value of the fifth feature amount is s₂₅ corresponding to the road type B. The reference value of the first feature amount is s₃₁, the reference value of the second feature amount is s₃₂, the reference value of the third feature amount is s₃₃, the reference value of the fourth feature amount is s₃₄, and the reference value of the fifth feature amount is s₃₅ corresponding to the road type C.

Next, the imaging device 4 will be described.

The imaging device 4 images at least a range where the face of the occupant of the vehicle 10 should be present. The imaging device 4 is a visible light camera or an infrared camera. In a case where the imaging device 4 is an infrared camera, the imaging device 4 is provided with a light source (not illustrated) that emits infrared rays for imaging to a range where the face of the occupant should be present.

The imaging device 4 outputs the captured image to the alertness level estimating device 2.

Next, the alertness level estimating device 2 will be described.

The alertness level estimating device 2 includes an image acquiring unit 21, a state detecting unit 22, a feature amount calculating unit 23, a road information acquiring unit 24, and an alertness level estimating unit 25.

The image acquiring unit 21 acquires a captured image from the imaging device 4.

The image acquiring unit 21 outputs the acquired captured image to the state detecting unit 22.

The state detecting unit 22 detects the state of the occupant on the basis of the captured image output from the image acquiring unit 21.

In the first embodiment, the state of the occupant detected by the state detecting unit 22 is a state of the occupant for calculating a feature amount used for estimating the alertness level of the occupant. Specifically, the state of the occupant detected by the state detecting unit 22 is the eyelid opening degree of the occupant, the mouth opening degree of the occupant, the face direction of the occupant, the head position of the occupant, the line-of-sight direction of the occupant, or the like. The state detecting unit 22 just needs to detect the state of the occupant using a known image recognition technology.

The state detecting unit 22 outputs the captured image to the feature amount calculating unit 23 in association with information regarding the detected state of the occupant. The captured image that is output from the state detecting unit 22 and associated with the information regarding the state of the occupant is also referred to as a “captured image after state detection”. The function of the state detecting unit 22 may be included in the feature amount calculating unit 23 described later.

The feature amount calculating unit 23 calculates the feature amount of the occupant on the basis of the captured image after state detection output from the state detecting unit 22.

The feature amount calculating unit 23 calculates the feature amount of the occupant for each frame of the captured image after state detection output from the state detecting unit 22. Note that, in a case where the feature amount is a feature amount that needs to be determined from the history of the state of the occupant in the past, such as the PERCLOS, the number of blinks, or the blink speed, the feature amount calculating unit 23 stores, for example, the captured image after state detection output from the state detecting unit 22, and calculates the feature amount on the basis of the stored captured image after state detection for a past preset number of frames (hereinafter referred to as “the number of frames for feature amount calculation”). For example, the feature amount calculating unit 23 may calculate the feature amount on the basis of a stored captured image after state detection acquired in a past preset period (hereinafter referred to as a “feature amount calculation target period”).

For example, in a case where the information regarding the state of the occupant associated with the captured image after state detection of one frame output from the state detecting unit 22 can be used as the feature amount of the occupant as it is, such that the feature amount is the eyelid opening degree of the occupant, the mouth opening degree of the occupant, the face direction of the occupant, the line-of-sight direction of the occupant, or the like, the feature amount calculating unit 23 just needs to use the information regarding the state of the occupant as the feature amount.

The feature amount calculating unit 23 outputs feature amount information in which the calculated feature amount is associated with the captured image to the alertness level estimating unit 25 and the reference value creating device 1.

The road information acquiring unit 24 acquires road information output from the reference value creating device 1.

The road information acquiring unit 24 outputs the acquired road information to the alertness level estimating unit 25.

The alertness level estimating unit 25 estimates the alertness level of the occupant on the basis of the road information output from the road information acquiring unit 24, the reference value of the feature amount created by the reference value creating device 1, and the feature amount included in the feature amount information output from the feature amount calculating unit 23. The reference value of the feature amount created by the reference value creating device 1 is stored in the storage unit 15 as the reference value information.

Specifically, the alertness level estimating unit 25 first refers to the storage unit 15 and acquires the reference value corresponding to the road type indicated by the road information output from the road information acquiring unit 24. Then, the alertness level estimating unit 25 calculates a difference between the feature amount included in the feature amount information and the acquired reference value according to the following Expression (1). The difference is, that is, a feature amount that absorbs individual differences.

Feature amount absorbing individual difference=feature amount-reference value (1)

Note that the feature amount included in the feature amount information output from the feature amount calculating unit 23 is a feature amount calculated on the basis of the captured image of the latest frame (current frame).

The alertness level estimating unit 25 calculates, for each feature amount, a difference from a reference value corresponding to the feature amount.

Then, the alertness level estimating unit 25 estimates the alertness level of the occupant on the basis of the calculated difference and a learned model (hereinafter referred to as a “machine learning model”).

The machine learning model is a machine learning model such as a support vector machine (SVM), a random forest, Light Gradient Boosting Machine (LightGBM), or a convolutional neural network. The machine learning model uses a difference between the feature amount and the reference value of the feature amount as an input, and outputs information regarding the alertness level. The machine learning model is created in advance by an administrator or the like and stored in a place that can be referred to by the alertness level estimating unit 25.

For example, it is assumed that the alertness level is represented on a scale of five, and the greater the number, the higher the alertness level. In this case, the alertness level estimating unit 25 inputs the calculated difference to the machine learning model and obtains the alertness level of “1” to “5”.

The alertness level estimating unit 25 is not limited to the method using the machine learning model, and may estimate the alertness level of the occupant on the basis of a preset condition (hereinafter referred to as an “alertness level estimation condition”).

As the alertness level estimation condition, for example, the following condition is set in advance. Note that the following example of the alertness level estimation condition is an example of the alertness level estimation condition in a case where the alertness level is expressed on a scale of five, and the higher the alertness level is, the more alert the user is.

### <Alertness Level Estimation Condition>

In a case where the difference from the reference value is equal to or less than a preset threshold value in all the feature amounts, the alertness level is “5”.
In a case where the ratio of the feature amount in which the difference from the reference value is equal to or less than a preset threshold is 20% or less in all the feature amounts, the alertness level is “1”
In a case where the ratio of the feature amount in which the difference from the reference value is equal to or less than the preset threshold is more than 20% and equal to or less than 40% in all the feature amounts, the alertness level is “2”.
In a case where the ratio of the feature amount in which the difference from the reference value is equal to or less than the preset threshold is more than 40% and equal to or less than 60% in all the feature amounts, the alertness level is “3”.
In a case where the ratio of the feature amount in which the difference from the reference value is equal to or less than the preset threshold is more than 60% and equal to or less than 80% in all the feature amounts, the alertness level is “4”.
In a case where the ratio of the feature amount in which the difference from the reference value is equal to or less than the preset threshold is more than 80% in all the feature amounts, the alertness level is “5”.

Note that, in the above example, the alertness level is represented by five levels, but this is merely an example. For example, the alertness level may be represented by “high” or “low”. In this case, the machine learning model uses the difference between the feature amount and the reference value of the feature amount as an input, and outputs information indicating “high” or “low”. In addition, as the alertness level estimation condition, a condition with which the alertness level can be estimated to be “high” or “low” is set on the basis of the difference between the feature amount and the reference value of the feature amount.

The alertness level estimating unit 25 outputs information (hereinafter referred to as “alertness level information”) regarding the estimated alertness level of the occupant to, for example, a driving assistance device (not illustrated). The alertness level information includes at least the alertness level. The alertness level information may include, for example, a seat position of the occupant or the captured image included in the feature amount information. It is assumed that the information regarding the seat position of the occupant is given to the captured image. For example, when detecting the state of the occupant, the state detecting unit 22 also detects the seat position of the occupant and gives the seat position to the captured image.

On the basis of the alertness level information, for example, when the alertness level of the occupant is equal to or less than a preset threshold, the driving assistance device outputs a warning sound from an output device (not illustrated) mounted on the vehicle 10.

An operation of the reference value creating device 1 according to the first embodiment will be described.

FIG. 3 is a flowchart for explaining the operation of the reference value creating device 1 according to the first embodiment.

Note that, when the power of the vehicle 10 is turned on, the reference value creating device 1 repeats the operation illustrated in the flowchart of FIG. 3 until the power of the vehicle 10 is turned off.

The vehicle-related information acquiring unit 11 acquires the vehicle-related information output from the vehicle-related information acquiring device 3 (step ST1).

The vehicle-related information acquiring unit 11 outputs the acquired vehicle-related information to the road determining unit 12.

The road determining unit 12 determines the road type of the road on which the vehicle 10 is traveling on the basis of the vehicle-related information acquired by the vehicle-related information acquiring unit 11 in step ST1 (step ST2).

The road determining unit 12 outputs the road information to the reference value creating unit 14 and the alertness level estimating device 2.

The feature amount acquiring unit 13 acquires the feature amount information from the alertness level estimating device 2 (step ST3).

The feature amount acquiring unit 13 outputs the acquired feature amount information to the reference value creating unit 14.

The reference value creating unit 14 performs “reference value creation processing” for creating a reference value for each feature amount on the basis of the road type determined by the road determining unit 12 in step ST2 and the feature amount information acquired by the feature amount acquiring unit 13 in step ST3 (step ST4).

When creating the reference value in the “reference value creation processing”, the reference value creating unit 14 stores the reference value information in the storage unit 15.

Note that, in the flowchart illustrated in FIG. 3, the reference value creating device 1 performs processing in the order of step ST1, step ST2, and step ST3, but the order of the processing of steps ST1 to ST3 is not limited thereto. The reference value creating device 1 just needs to perform the processing of step ST3 before performing the processing of step ST4.

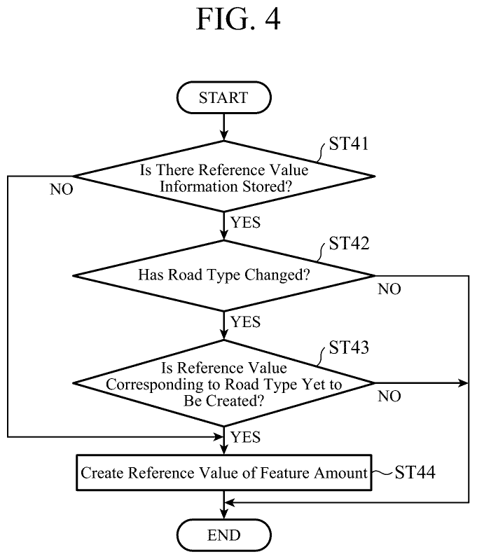

FIG. 4 is a flowchart for explaining a specific operation of step ST4 in FIG. 3.

The reference value creating unit 14 determines whether or not the reference value information is stored in the storage unit 15 (step ST41).

When it is determined in step ST41 that the reference value information is stored in the storage unit 15 (when “YES” in step ST41), the reference value creating unit 14 determines whether or not the road type of the road on which the vehicle 10 is traveling has changed on the basis of the road information output from the road determining unit 12 in step ST2 of FIG. 3 (step ST42).

When it is determined in step ST42 that the road type has changed (when “YES” in step ST42), the reference value creating unit 14 refers to the storage unit 15 and determines whether or not a reference value corresponding to the changed road type is yet to be created (step ST43).

When it is determined in step ST43 that the reference value corresponding to the changed road type is yet to be created (when “YES” in step ST43), or when it is determined in step ST41 that the reference value information is not stored in the storage unit 15 (when “NO” in step ST41), the reference value creating unit 14 creates the reference value for each feature amount on the basis of the feature amount information acquired by the feature amount acquiring unit 13 in step ST3 of FIG. 3 (step ST44). Then, the reference value creating unit 14 stores the reference value information in the storage unit 15.

When it is determined in step ST42 that the road type has not changed (when “NO” in step ST42), or when it is determined in step ST43 that the reference value corresponding to the changed road type has been created (when “NO” in step ST43), the reference value creating device 1 ends the process illustrated in the flowchart of FIG. 4.

In the determination in step ST41 described above, the reference value creating unit 14 determines whether (the first condition) of the reference value creation condition is satisfied. In other words, the reference value creating unit 14 determines whether it is when the power of the vehicle 10 is turned on and the vehicle 10 starts traveling in the determination of step ST41 described above.

Further, in the determination in steps ST42 to ST43 described above, the reference value creating unit 14 determines whether (the second condition) of the reference value creation condition is satisfied.

Note that, in the first embodiment, the reference value creating unit 14 determines the above (first condition), that is, whether or not it is when the power of the vehicle 10 is turned on and the vehicle 10 starts traveling depending on whether or not the reference value information is stored in the storage unit 15 (see step ST41 described above), but this is merely an example. For example, the reference value creating unit 14 may determine that it is when the power of the vehicle 10 is turned on and the vehicle 10 starts traveling by, for example, a travel start flag. The travel start flag is stored in a place that can be referred to by the reference value creating unit 14. When creating the reference value, the reference value creating unit 14 sets “1” to the travel start flag. The travel start flag is initialized to “0” when the power of the vehicle 10 is turned off. When the travel start flag is “0”, the reference value creating unit 14 determines that the reference value has not been created yet when the power of the vehicle 10 is turned on and the vehicle 10 starts to travel, that is, after the power of the vehicle 10 is turned on.

As described above, when the reference value creating unit 14 determines, for example, by the travel start flag that it is when the power of the vehicle 10 is turned on and the vehicle 10 starts to travel, the reference value information stored in the storage unit 15 is not necessarily deleted when the power of the vehicle 10 is turned off.

Further, in a case where the road type of the road on which the vehicle 10 is traveling is changed and the reference value for the road type is yet to be created, and thus the reference value is to be created, that is, in a case where it is time to create the reference value corresponding to the changed road type, the reference value creating unit 14 creates the reference value after the feature amount associated with the captured image in the feature amount information becomes the feature amount calculated on the basis of the captured image captured in the vehicle 10 traveling on the road after the road type is changed. Describing with a specific example, for example, it is assumed that the feature amount calculating unit 23 of the alertness level estimating device 2 calculates the feature amount on the basis of the captured images after state detection for the number of frames for feature amount calculation. In addition, it is assumed that the reference value creating unit 14 creates the reference value from the feature amounts included in the feature amount information corresponding to the captured images for the number of frames for reference value creation. In this case, after determining that the road type has changed, the reference value creating unit 14 waits without creating the reference value while the feature amount information for the number of frames for feature amount calculation is output from the feature amount acquiring unit 13. This is because the feature amount included in the feature amount information may be a feature amount calculated before the road type changes. Then, after the feature amount information for the number of frames for feature amount calculation is output from the feature amount acquiring unit 13, the reference value creating unit 14 creates a reference value from the feature amounts included in the feature amount information corresponding to the captured images for the number of frames for reference value creation acquired from the feature amount acquiring unit 13.

In addition, for example, it is assumed that the feature amount calculating unit 23 of the alertness level estimating device 2 calculates the feature amount on the basis of the captured image after state detection acquired in the feature amount calculation target period. In addition, it is assumed that the reference value creating unit 14 creates the reference value from the feature amount included in the feature amount information acquired in the reference value creation target period. In this case, the reference value creating unit 14 waits without creating the reference value until the feature amount calculation target period elapses. Then, after the feature amount calculation target period has elapsed, the reference value creating unit 14 creates a reference value from the feature amount included in the feature amount information corresponding to the captured image for the reference value creation target period acquired from the feature amount acquiring unit 13.

Note that, in a case where the feature amount is not a feature amount that needs to be determined from the background of the state of the occupant in the past, the reference value creating unit 14 may create the reference value without waiting after determining that the road type has changed.

The operation of the reference value creating device 1 described with reference to FIGS. 3 and 4 will be described with an example of a traveling status of the vehicle 10.

For example, it is assumed that an occupant gets into the vehicle 10, turns on the power of the vehicle 10, and starts traveling on an ordinary road. At this stage, since the reference value creating device 1 has never created a reference value, the reference value creating device 1 creates a reference value corresponding to “ordinary road” (when “NO” in step ST41 in FIG. 4, see step ST44). Then, the reference value creating device 1 stores reference value information in which information that can specify the “ordinary road” is associated with the created reference value.

Thereafter, it is assumed that the vehicle 10 travels on the ordinary road for a while. During this time, the reference value creating device 1 does not create the reference value (see the case of “YES” in step ST41 and the case of “NO” in step ST42 in FIG. 4).

It is assumed that the vehicle 10 enters an expressway after traveling on the ordinary road for a while. Then, the reference value creating device 1 determines that the road type has changed. Here, at the time of traveling on the expressway, the reference value has not been created yet. Therefore, the reference value creating device 1 creates the reference value corresponding to “expressway” (see the case of “YES” in step ST41, the case of “YES” in step ST42, and the case of “YES” in step ST43 in FIG. 4). Then, the reference value creating device 1 stores reference value information in which information that can specify the “expressway” is associated with the created reference value.

It is assumed that after traveling on the expressway for a while, the vehicle 10 gets off the expressway and enters an ordinary road again. Then, the reference value creating device 1 determines that the road type has changed. Here, at the time of traveling on the ordinary road, the reference value has already been created. Therefore, the reference value creating device 1 does not create the reference value (see the case of “YES” in step ST41, the case of “YES” in step ST42, and the case of “NO” in step ST43 in FIG. 4).

As described above, in the reference value creating device 1, even when the road type of the road on which the vehicle 10 is traveling changes, if the reference value for the changed road type has been created, the reference value creating unit 14 determines that “the second condition” is not satisfied and does not create the reference value. In other words, when the road type has changed, the reference value creating unit 14 does not create the reference value when the reference value information in which the changed road type and the reference value are associated with each other is stored in the storage unit 15. That is, the reference value creating unit 14 does not update the created reference value if the reference value has been created in the same road type in the past after the power of the vehicle 10 is turned on.

Further, the reference value creating unit 14 creates the reference value corresponding to the road type of the road on which the vehicle 10 is traveling once when the power of the vehicle 10 is turned on, and thereafter, creates the reference value corresponding to the changed road type only once when the road type on which the vehicle 10 is traveling is changed.

For example, when an occupant drives the vehicle 10, the occupant is in an awake state immediately after starting driving, but if driving on a road of the same road type continues, it is assumed that the alertness level gradually decreases due to fatigue, habituation, or the like. Therefore, the reference value used for estimating the alertness level of the occupant is preferably created immediately after the start of driving, at which it is assumed to be in the awake state. This is because, when the reference value is created from the feature amount based on the state of the occupant immediately after the start of driving, a difference between the reference value and the feature amount when the alertness level of the occupant decreases occurs more clearly, and it is considered that the alertness level of the occupant can be detected with high accuracy. When the reference value is created in a state where the alertness level is low, the reference value may be lower than the reference value that is supposed to be created. When the reference value becomes lower than the reference value supposed to be created, the difference between the reference value and the feature amount when the alertness level of the occupant decreases is less likely to occur clearly.

However, since the road type affects the feature amount, it is necessary to review the reference value when the road type changes. This is because, depending on the feature amount, the change depending on the road type is large, and even if the alertness level of the occupant does not change, the difference from the reference value increases due to the change in the road type. When the reference value is created without considering a change in the feature amount depending on the road type, and the reference value is compared with the feature amount when the road type has changed, for example, a difference between the reference value and the feature amount increases even though the alertness level of the occupant has not decreased, and erroneous detection of the alertness level of the occupant occurs.

Therefore, the reference value creating device 1 creates the reference value once when the power of the vehicle 10 is turned on, and once when the road type is changed and it is determined that it is necessary to review the reference value. At that time, the reference value creating device 1 creates the reference value corresponding to the changed road type on the basis of the feature amount information regarding the feature amount calculated on the basis of the captured image captured in the vehicle 10 traveling on the road of the changed road type. Thus, the reference value creating device 1 can create the reference value for estimating the alertness level of the occupant in consideration of an individual difference in the tendency of a change in the feature amount depending on the road type.

The operation of the alertness level estimating device 2 according to the first embodiment will be described.

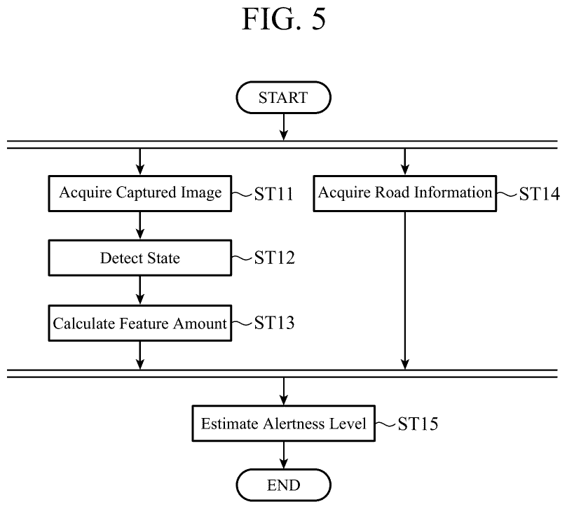

FIG. 5 is a flowchart for explaining the operation of the alertness level estimating device 2 according to the first embodiment.

The image acquiring unit 21 acquires a captured image from the imaging device 4 (step ST11).

The image acquiring unit 21 outputs the acquired captured image to the state detecting unit 22.

The state detecting unit 22 detects the state of the occupant on the basis of the captured image output from the image acquiring unit 21 in step ST11 (step ST12).

The state detecting unit 22 outputs the captured image after state detection to the feature amount calculating unit 23.

The feature amount calculating unit 23 calculates the feature amount of the occupant on the basis of the captured image after state detection output from the state detecting unit 22 in step ST12 (step ST13).

The feature amount calculating unit 23 outputs the feature amount information to the alertness level estimating unit 25 and the reference value creating device 1.

The road information acquiring unit 24 acquires road information output from the reference value creating device 1 (step ST14).

The road information acquiring unit 24 outputs the acquired road information to the alertness level estimating unit 25.

The alertness level estimating unit 25 estimates the alertness level of the occupant on the basis of the road information output from the road information acquiring unit 24 in step ST14, the reference value created by the reference value creating device 1, and the feature amount included in the feature amount information output from the feature amount calculating unit 23 in step ST13 (step ST15). The reference value created by the reference value creating device 1 is stored in the storage unit 15 as the reference value information. The alertness level estimating unit 25 compares the road information acquired by the road information acquiring unit 24 in step ST14 with the reference value information stored in the storage unit 15, and acquires the reference value corresponding to the road type determined by the reference value creating device 1 from the storage unit 15. Then, the alertness level estimating unit 25 estimates the alertness level of the occupant using the acquired reference value.

Specifically, the alertness level estimating unit 25 estimates the alertness level of the occupant on the basis of the difference between the feature amount calculated by the feature amount calculating unit 23 and the reference value of the feature amount and the machine learning model. Alternatively, the alertness level estimating unit 25 estimates the alertness level of the occupant on the basis of the difference between the feature amount calculated by the feature amount calculating unit 23 and the reference value of the feature amount and the alertness level estimation condition.

Note that, in a case where the road type has changed and the reference value information including the reference value corresponding to the changed road type is not yet stored in the storage unit 15, the alertness level estimating unit 25 estimates the alertness level of the occupant using the reference value corresponding to the previous road type, in other words, the reference value used until the road type is changed. For example, the alertness level estimating unit 25 just needs to store the road information acquired from the road information acquiring unit 24 for a constant period of time and determine whether or not the road type has changed from the stored road information.

Further, in a case where the reference value information is not stored in the storage unit 15, the alertness level estimating unit 25 does not estimate the alertness level of the occupant until the reference value information is stored. The case where the reference value information is not stored in the storage unit 15 is assumed to be, for example, a period until the reference value is first created in the reference value creating device 1 after the power of the vehicle 10 is turned on and the reference value information is stored in the storage unit 15.

The alertness level estimating unit 25 outputs the alertness level information to, for example, the driving assistance device.

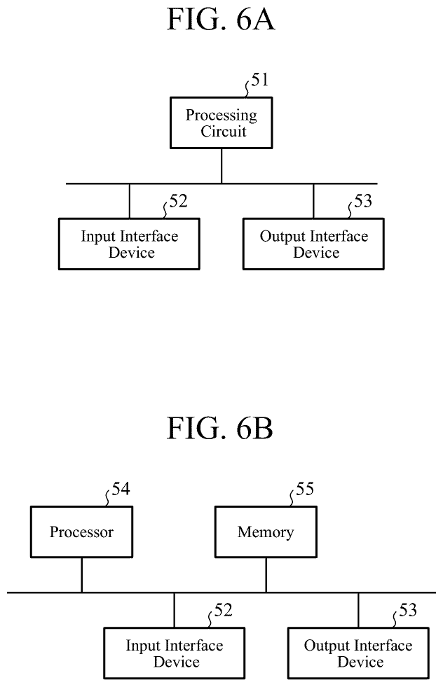

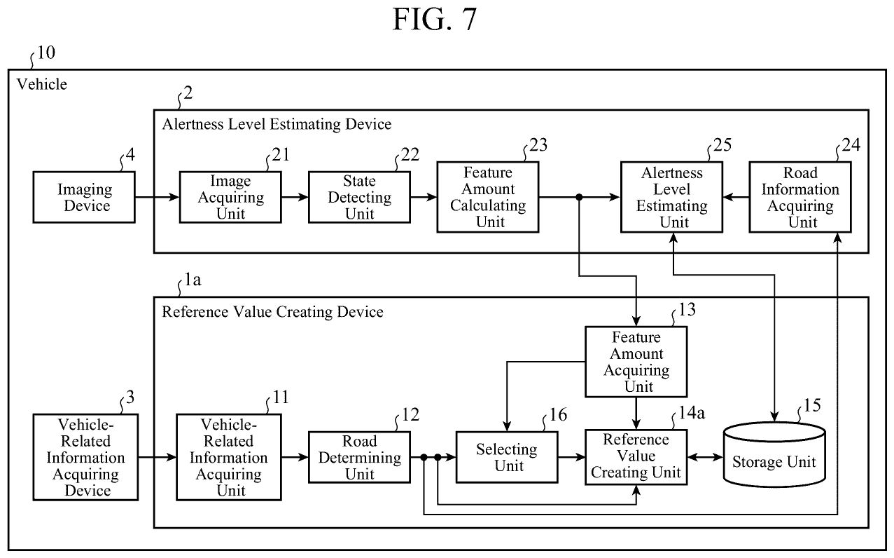

FIGS. 6A and 6B are diagrams illustrating an example of a hardware configuration of the reference value creating device 1 according to the first embodiment.

In the first embodiment, the functions of the vehicle-related information acquiring unit 11, the road determining unit 12, the feature amount acquiring unit 13, and the reference value creating unit 14 are implemented by a processing circuit 51. That is, the reference value creating device 1 includes the processing circuit 51 for creating the reference value of the feature amount used for estimating the alertness level of the occupant on the basis of the vehicle-related information and the feature amount information.

The processing circuit 51 may be dedicated hardware as illustrated in FIG. 6A or a processor 54 that executes a program stored in a memory as illustrated in FIG. 6B.

In a case where the processing circuit 51 is dedicated hardware, the processing circuit 51 corresponds to, for example, a single circuit, a composite circuit, a programmed processor, a parallel programmed processor, an application specific integrated circuit (ASIC), a field-programmable gate array (FPGA), or a combination thereof.

When the processing circuit is the processor 54, the functions of the vehicle-related information acquiring unit 11, the road determining unit 12, the feature amount acquiring unit 13, and the reference value creating unit 14 are implemented by software, firmware, or a combination of software and firmware. Software or firmware is described as a program and stored in a memory 55. The processor 54 reads and executes the program stored in the memory 55, thereby executing the functions of the vehicle-related information acquiring unit 11, the road determining unit 12, the feature amount acquiring unit 13, and the reference value creating unit 14. That is, the reference value creating device 1 includes the memory 55 for storing a program that results in execution of steps ST1 to ST5 in FIG. 3 described above when executed by the processor 54. Further, it can also be said that the program stored in the memory 55 causes a computer to execute a procedure or a method performed by processing of the vehicle-related information acquiring unit 11, the road determining unit 12, the feature amount acquiring unit 13, and the reference value creating unit 14. Here, the memory 55 corresponds to, for example, a nonvolatile or volatile semiconductor memory such as a RAM, a read only memory (ROM), a flash memory, an erasable programmable read only memory (EPROM), or an electrically erasable programmable read-only memory (EEPROM), or a magnetic disk, a flexible disk, an optical disk, a compact disk, a mini disk, a digital versatile disc (DVD), or the like.

Note that the functions of the vehicle-related information acquiring unit 11, the road determining unit 12, the feature amount acquiring unit 13, and the reference value creating unit 14 may be partially implemented by dedicated hardware and partially implemented by software or firmware. For example, the functions of the vehicle-related information acquiring unit 11 and the feature amount acquiring unit 13 can be implemented by the processing circuit 51 as dedicated hardware, and the functions of the road determining unit 12 and the reference value creating unit 14 can be implemented by the processor 54 reading and executing a program stored in the memory 55.

Further, the reference value creating device 1 includes an input interface device 52 and an output interface device 53 that perform wired communication or wireless communication with a device such as the alertness level estimating device 2 or the vehicle-related information acquiring device 3.

Furthermore, the storage unit 15 uses the memory 55. Note that this is an example, and the storage unit 15 may be configured by an HDD, a solid state drive (SSD), a DVD, or the like.

A diagram illustrating an example of a hardware configuration of the alertness level estimating device 2 according to the first embodiment is similar to FIGS. 6A and 6B illustrating an example of a hardware configuration of the reference value creating device 1.

In the first embodiment, the functions of the image acquiring unit 21, the state detecting unit 22, the feature amount calculating unit 23, the road information acquiring unit 24, and the alertness level estimating unit 25 are implemented by the processing circuit 51. That is, the alertness level estimating device 2 includes the processing circuit 51 for estimating the alertness level of the occupant on the basis of the captured image.

The processing circuit 51 may be dedicated hardware as illustrated in FIG. 6A or the processor 54 that executes a program stored in a memory as illustrated in FIG. 6B

In a case where the processing circuit 51 is dedicated hardware, the processing circuit 51 corresponds to, for example, a single circuit, a composite circuit, a programmed processor, a parallel programmed processor, an application specific integrated circuit (ASIC), a field-programmable gate array (FPGA), or a combination thereof.

When the processing circuit is the processor 54, the functions of the image acquiring unit 21, the state detecting unit 22, the feature amount calculating unit 23, the road information acquiring unit 24, and the alertness level estimating unit 25 are implemented by software, firmware, or a combination of software and firmware. The software or firmware is described as a program and stored in the memory 55. The processor 54 reads and executes the program stored in the memory 55, thereby executing the functions of the image acquiring unit 21, the state detecting unit 22, the feature amount calculating unit 23, the road information acquiring unit 24, and the alertness level estimating unit 25. That is, the alertness level estimating device 2 includes the memory 55 for storing programs that, when executed by the processor 54, result in execution of steps ST11 to ST15 of FIG. 4 described above. Further, it can also be said that the program stored in the memory 55 causes a computer to execute a procedure or a method performed by processing of the image acquiring unit 21, the state detecting unit 22, the feature amount calculating unit 23, the road information acquiring unit 24, and the alertness level estimating unit 25. Here, the memory 55 corresponds to, for example, a nonvolatile or volatile semiconductor memory such as a RAM, a read only memory (ROM), a flash memory, an erasable programmable read only memory (EPROM), or an electrically erasable programmable read-only memory (EEPROM), or a magnetic disk, a flexible disk, an optical disk, a compact disk, a mini disk, a digital versatile disc (DVD), or the like.

Note that the functions of the image acquiring unit 21, the state detecting unit 22, the feature amount calculating unit 23, the road information acquiring unit 24, and the alertness level estimating unit 25 may be partially implemented by dedicated hardware and partially implemented by software or firmware. For example, the functions of the image acquiring unit 21 and the road information acquiring unit 24 can be implemented by the processing circuit 51 as dedicated hardware, and the functions of the state detecting unit 22, the feature amount calculating unit 23, and the alertness level estimating unit 25 can be implemented by the processor 54 reading and executing the program stored in the memory 55.

Further, the alertness level estimating device 2 includes the input interface device 52 and the output interface device 53 that perform wired communication or wireless communication with a device such as the reference value creating device 1, the imaging device 4, or the driving assistance device.

Note that, in the first embodiment described above, when the power source of the vehicle 10 is turned off, the reference value information stored in the storage unit 15 is deleted. However, this is merely an example, and the reference value information stored in the storage unit 15 may not be deleted. In this case, instead of the (first condition) as described above, for example, “the reference value information corresponding to the occupant is stored in the storage unit 15 (third condition)” is set as the reference value creation condition. In step ST41 of FIG. 4, the reference value creating unit 14 determines whether or not the reference value information corresponding to the occupant is stored in the storage unit 15.

The reference value information stored in the storage unit 15 by the reference value creating unit 14 is associated with information that can specify the occupant. For example, when the state detecting unit 22 detects the state of the occupant, personal authentication is performed, and the authentication result is included in the feature amount information. The reference value creating unit 14 acquires the authentication result via the feature amount acquiring unit 13, and stores the authentication result in the storage unit 15 in association with the reference value information. In addition, for example, when the occupant gets into the vehicle, the occupant may operate an input device (not illustrated) mounted on the vehicle 10 to input personal information, and the reference value creating unit 14 may receive the input personal information and store the personal information in the storage unit 15 in association with the reference value information. The input device is assumed to be, for example, a touch panel display mounted on the vehicle 10.

Note that, as a meaning of deleting the reference value information stored in the storage unit 15 when the power of the vehicle 10 is turned off, for example, the reference value creating device 1 can be effectively operated without specifying an occupant including an occupant who drives the vehicle 10 for the first time.

Further, in the first embodiment described above, the alertness level estimating unit 25 of the alertness level estimating device 2 does not estimate the alertness level of the occupant until the reference value creating device 1 creates the reference value for the first time after the power of the vehicle 10 is turned on and the reference value information is stored in the storage unit 15, but this is merely an example. For example, a general reference value used only until the reference value creating unit 14 stores the reference value information in the reference value creating device 1 is stored in the storage unit 15, and the alertness level estimating unit 25 may estimate the alertness level of the occupant using the general reference value. In the reference value creating device 1, when the reference value information is stored in the storage unit 15 for the first time, the reference value creating unit 14 notifies the alertness level estimating unit 25 of the fact. Upon receiving the notification from the reference value creating unit 14, the alertness level estimating unit 25 stops using the general reference value.

In the first embodiment described above, the reference value creating device 1 and the alertness level estimating device 2 are separate devices and are each mounted on the vehicle 10, but this is merely an example. For example, the reference value creating device 1 may be mounted on the alertness level estimating device 2, or the alertness level estimating device 2 may be mounted on the reference value creating device 1. Further, in the first embodiment described above, the image acquiring unit 21, the state detecting unit 22, and the feature amount calculating unit 23 are included in the alertness level estimating device 2, but this is merely an example. For example, the image acquiring unit 21, the state detecting unit 22, and the feature amount calculating unit 23 may be provided in the reference value creating device 1, or may be provided in other places that can be referred to by the alertness level estimating device 2 and the reference value creating device 1.

Further, in the first embodiment described above, the reference value creating device 1 and the alertness level estimating device 2 are in-vehicle devices mounted on the vehicle 10, and the vehicle-related information acquiring unit 11, the road determining unit 12, the feature amount acquiring unit 13, the reference value creating unit 14, the image acquiring unit 21, the state detecting unit 22, the feature amount calculating unit 23, the road information acquiring unit 24, and the alertness level estimating unit 25 are included in the in-vehicle device. Not limited to this, some of the vehicle-related information acquiring unit 11, the road determining unit 12, the feature amount acquiring unit 13, the reference value creating unit 14, the image acquiring unit 21, the state detecting unit 22, the feature amount calculating unit 23, the road information acquiring unit 24, and the alertness level estimating unit 25 may be included in an in-vehicle device of a vehicle, and others may be included in a server connected to the in-vehicle device via a network.

Further, in the first embodiment described above, the occupant whose alertness level is to be estimated is a driver, but this is merely an example. The occupants whose alertness level is to be estimated include an occupant in the passenger seat or an occupant in the rear seat. Furthermore, a plurality of occupants may be targets for estimating the alertness level. In this case, the reference value creating device 1 creates a reference value for each occupant, and causes the storage unit 15 to store reference value information for each occupant. Further, the alertness level estimating device 2 estimates the alertness level for each occupant on the basis of the reference values corresponding to the occupant and the road type of the road on which the vehicle 10 is traveling.

As described above, according to the first embodiment, the reference value creating device 1 includes the vehicle-related information acquiring unit 11 to acquire vehicle-related information related to the vehicle 10, the road determining unit 12 to determine a road type of a road on which the vehicle 10 is traveling on the basis of the vehicle-related information acquired by the vehicle-related information acquiring unit 11, the feature amount acquiring unit 13 to acquire feature amount information regarding a feature amount of an occupant of the vehicle 10 calculated on the basis of a captured image capturing an image of a range in which at least the face of the occupant is present, and the reference value creating unit 14 to create the reference value of the feature amount for estimating the alertness level of the occupant on the basis of the feature amount information acquired by the feature amount acquiring unit 13, and create, when the road type determined by the road determining unit 12 has changed, the reference value of the feature amount corresponding to the changed road type on the basis of the feature amount information regarding the feature amount calculated on the basis of the captured image captured in the vehicle 10 traveling on the road of the changed road type. Therefore, the reference value creating device 1 can create a reference value for estimating the alertness level of the occupant in consideration of individual differences in the tendency of a change in the feature amount depending on the road type.

Further, according to the first embodiment, the alertness level estimating device 2 includes the road information acquiring unit 24 to acquire road information regarding the road type determined by the reference value creating device 1, and the alertness level estimating unit 25 to estimate the alertness level of the occupant on the basis of the reference value of the feature amount corresponding to the road type indicated by the road information acquired by the road information acquiring unit 24 and the feature amount, the reference value being created by the reference value creating device 1. Since the alertness level estimating device 2 estimates the alertness level of the occupant using the reference value created in consideration of the individual difference in the tendency of a change in the feature amount depending on the road type, it is possible to accurately estimate the alertness level of the occupant as compared with a case where the reference value is not created in consideration of the individual difference in the tendency of a change in the feature amount depending on the road type.

### Second Embodiment

In the first embodiment, when the road type of the road on which the vehicle is traveling is changed and it is determined that it is time to create the reference value corresponding to the changed road type, the reference value creating device creates the reference value for all the feature amounts.

In a second embodiment, an embodiment will be described in which, when a reference value creating device determines that it is time to create a reference value corresponding to a changed road type, a feature amount to be created is selected again, and the reference value is created only for the selected feature amount.

FIG. 7 is a diagram illustrating a configuration example of a reference value creating device 1a and an alertness level estimating device 2 according to the second embodiment.

In the configuration example of the reference value creating device 1a according to the second embodiment, the same reference numerals are given to configuration examples similar to those of the reference value creating device 1 described with reference to FIG. 1 in the first embodiment, and redundant description will be omitted. The reference value creating device 1a according to the second embodiment is different from the reference value creating device 1 according to the first embodiment in that a selecting unit 16 is included. Further, in the second embodiment, a specific operation of a reference value creating unit 14a of the reference value creating device 1a is different from the specific operation of the reference value creating unit 14 of the reference value creating device 1 according to the first embodiment.

The configuration example of the alertness level estimating device 2 according to the second embodiment is similar to the configuration example of the alertness level estimating device 2 according to the first embodiment, and thus the same reference numerals are given to omit redundant description.

When the road type of the road on which the vehicle 10 is traveling which is determined by the road determining unit 12 has changed, the selecting unit 16 selects a feature amount (hereinafter referred to as a “target feature amount”) for which a reference value corresponding to the changed road type is to be created from among the feature amounts (first feature amount, second feature amount, third feature amount, fourth feature amount, and fifth feature amount) included in the feature amount information acquired by the feature amount acquiring unit 13.

Note that, in the second embodiment, the road determining unit 12 outputs the road information to the selecting unit 16, the reference value creating unit 14a, and the alertness level estimating device 2. Further, in the second embodiment, the feature amount acquiring unit 13 outputs the feature amount information to the selecting unit 16 and the reference value creating unit 14a.

An example of a specific method in which the selecting unit 16 selects the target feature amount will be described.

### Specific Example 1

For example, information (hereinafter referred to as “selection information”) in which a feature amount to be a target feature amount is defined when the road type is changed is created in advance and stored in the selecting unit 16. The selecting unit 16 selects the target feature amount according to the selection information. For example, assuming that the first feature amount is the mouth opening degree, the second feature amount is the movement of a mouth corner, the third feature amount is the face direction, the fourth feature amount is the line-of-sight direction, and the fifth feature amount is the blink speed, the mouth opening degree, the movement of the mouth corner, and the blink speed are relatively less affected by a change in the road type (here, whether it is an ordinary road or an expressway). On the other hand, the face direction and the line-of-sight direction are likely to be affected by the change in the road type. For example, it is difficult to assume that the action of opening the mouth or the speed of blinking of the occupant increases rapidly on an ordinary road or increases rapidly on an expressway. However, in an ordinary road where there are many signals or there is a possibility of running out in the street, the occupant often faces upward or checks the surroundings, and in an expressway, movement of the face and the line of sight of the occupant is less than that in the ordinary road. That is, among the mouth opening degree, the movement of the mouth corner, the face direction, the line-of-sight direction, and the blinking speed, the face direction and the line-of-sight direction are easily affected by the change in the road type, and it can be said that the individual difference in the tendency of the change in the feature amount depending on the road type more easily occurs.

In the selection information, for example, the face direction and the line-of-sight direction are defined as the target feature amount. The selecting unit 16 selects the third feature amount that is the face direction and the fourth feature amount that is the line-of-sight direction as the target feature amounts according to the selection information.

Note that, depending on the road type, it is also conceivable that there is a feature amount that is affected because of the type. Accordingly, for example, the selection information may be information in which information that can specify the changed road type is associated with the feature amount to be the target feature amount. The selecting unit 16 specifies the target feature amount by comparing the changed road type with the road type of the selection information.

### Specific Example 2

For example, the selecting unit 16 determines whether or not the alertness level of the occupant estimated in a preset period (hereinafter referred to as a “change determination period”) before the road type changes tends to decrease. For example, in the alertness level estimating device 2, the alertness level estimating unit 25 just needs to assign the time when estimation is performed to information of the estimated alertness level of the occupant and store the information in the storage unit 15, and the selecting unit 16 just needs to acquire the alertness level of the occupant estimated in the change determination period from the storage unit 15. The selecting unit 16 selects all the feature amounts as the target feature amount in a case where the alertness level of the occupant estimated in the change determination period does not tend to decrease, and selects only the feature amount that is likely to be affected by a change in the road type as the target feature amount in a case where the alertness level of the occupant tends to decrease. The selecting unit 16 just needs to specify a feature amount that is likely to be affected by the change in the road type from, for example, the above-described selection information.

For example, the first feature amount is the mouth opening degree, the second feature amount is the movement of the mouth corner, the third feature amount is the face direction, the fourth feature amount is the line-of-sight direction, and the fifth feature amount is the blink speed. Then, it is assumed that the road on which the vehicle 10 is traveling is changed from an ordinary road to an expressway. Here, if the alertness level of the occupant estimated in the change determination period going back from the time point at which the road type changes does not tend to decrease, the selecting unit 16 selects the first feature amount, the second feature amount, the third feature amount, the fourth feature amount, and the fifth feature amount as the target feature amounts. If the alertness level of the occupant estimated in the change determination period going back from the time point at which the road type changes tends to decrease, the selecting unit 16 selects the third feature amount and the fourth feature amount as the target feature amounts. For example, in a case where it has been estimated that the state of the occupant has changed in the “sleepy” direction (the direction in which the alertness level decreases) in the change determination period going back from the time point at which the road type has changed, the selecting unit 16 determines that the tendency of the change in the feature amount due to the change in the state of the occupant in the “sleepy” direction has a larger influence on the alertness level estimation than the tendency of the change in the feature amount due to the change in the road type, and selects the third feature amount and the fourth feature amount as the target feature amount. That is, the selecting unit 16 reduces the target feature amount.

In the above example, the selecting unit 16 selects all the feature amounts as the target feature amount when the alertness level of the occupant does not tend to decrease, but this is merely an example. For example, in a case where the alertness level of the occupant does not tend to decrease, the selecting unit 16 may select some feature amounts (for example, third feature amount, fourth feature amount, and fifth feature amount) including the feature amount that is likely to be affected by the change in the road type among all the feature amounts as the target feature amounts. When the alertness level of the occupant does not tend to decrease, the selecting unit 16 increases the target feature amount more than when the alertness level of the occupant tends to decrease.

The selecting unit 16 outputs information regarding the target feature amount (hereinafter referred to as “target feature amount information”) to the reference value creating unit 14a.

The reference value creating unit 14a creates a reference value on the basis of the feature amount information acquired by the feature amount acquiring unit 13. The reference value creating unit 14a creates the reference value for each feature amount. The reference value creating unit 14 creates the reference value when the reference value creation condition is satisfied. Since the reference value creation condition has been described in the first embodiment, a detailed description thereof will be omitted.

In addition, a specific creation method when the reference value creating unit 14a creates the reference value is similar to the specific creation method when the reference value creating unit 14 creates the reference value described in the first embodiment, and thus a detailed description thereof will be omitted.

However, in the second embodiment, when the road type determined by the road determining unit 12 has changed and it is determined that the reference value corresponding to the changed road type should be created, the reference value creating unit 14a creates the reference value on the basis of the feature amount information output from the feature amount acquiring unit 13 for the target feature amount among feature amounts, and sets the reference value to the same value as the reference value created immediately before for feature amounts other than the target feature amount. The reference value creating unit 14a can determine the target feature amount on the basis of the target feature amount information output from the selecting unit 16. Specifically, the reference value creating unit 14a creates the reference value on the basis of the feature amount information regarding the feature amount calculated on the basis of the captured image captured in the vehicle 10 traveling on the road after the road type is changed for the target feature amount with respect to the reference value to be created. The reference value creating unit 14a sets, as the reference value, the same value as the reference value corresponding to the road type of the road on which the vehicle 10 has been traveling before the road type is changed, more specifically, immediately before the road type is changed for the feature amount other than the target feature amount. The reference value corresponding to the road type of the road on which the vehicle 10 has been traveling immediately before the road type is changed is a reference value used for estimating the alertness level of the occupant in the alertness level estimating device 2 immediately before the road type is changed.

In the first embodiment, when it is determined that the reference value corresponding to the changed road type should be created due to a change in the road type of the road on which the vehicle 10 is traveling, the reference value creating unit 14 creates the reference values for all the feature amounts of the first feature amount, the second feature amount, the third feature amount, the fourth feature amount, and the fifth feature amount on the basis of the feature amount information.

In the second embodiment, when it is determined that the reference value corresponding to the changed road type should be created due to a change in the road type of the road on which the vehicle 10 is traveling, the reference value creating unit 14a creates the reference value based on the feature amount information again for the target feature amount among the first feature amount, the second feature amount, the third feature amount, the fourth feature amount, and the fifth feature amount, and uses the reference value that has been used to estimate the alertness level of the occupant in the alertness level estimating device 2 so far for the reference value of the feature amount other than the target feature amount.

Note that the reference value creating unit 14a creates the reference values for all the feature amounts when it is the timing to create the reference value for the first time after the power of the vehicle 10 is turned on.

The reference value creating unit 14a stores the created reference value information in the storage unit 15.

An operation of the reference value creating device 1a according to the second embodiment will be described.

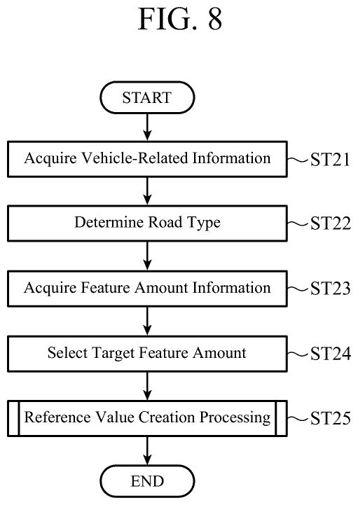

FIG. 8 is a flowchart for explaining the operation of the reference value creating device 1a according to the second embodiment.

Note that, when the power of the vehicle 10 is turned on, the reference value creating device 1a repeats the operation illustrated in the flowchart of FIG. 8 until the power of the vehicle 10 is turned off.

The vehicle-related information acquiring unit 11 acquires the vehicle-related information output from the vehicle-related information acquiring device 3 (step ST21). The specific operation of step ST21 is similar to the specific operation of step ST1 of FIG. 3 by the reference value creating device 1 described in the first embodiment.

The vehicle-related information acquiring unit 11 outputs the acquired vehicle-related information to the road determining unit 12.

The road determining unit 12 determines the road type of the road on which the vehicle 10 is traveling on the basis of the vehicle-related information acquired by the vehicle-related information acquiring unit 11 in step ST21 (step ST22). The specific operation of step ST22 is similar to the specific operation of step ST2 of FIG. 3 by the reference value creating device 1 described in the first embodiment.

The road determining unit 12 outputs the road information to the selecting unit 16, the reference value creating unit 14a, and the alertness level estimating device 2.

The feature amount acquiring unit 13 acquires the feature amount information from the alertness level estimating device 2 (step ST23). The specific operation of step ST23 is similar to the specific operation of step ST3 of FIG. 3 by the reference value creating device 1 described in the first embodiment.

The feature amount acquiring unit 13 outputs the acquired feature amount information to the reference value creating unit 14a and the selecting unit 16.

The selecting unit 16 selects a target feature amount when the road type determined by the road determining unit 12 in step ST22 has changed (step ST24).

The selecting unit 16 outputs the target feature amount information to the reference value creating unit 14a.

The reference value creating unit 14a performs “reference value creation processing” for creating a reference value for each feature amount on the basis of the road type determined by the road determining unit 12 in step ST22, the feature amount information acquired by the feature amount acquiring unit 13 in step ST23, and the target feature amount information output from the selecting unit 16 in step ST24 (step ST25).

When creating the reference value in the “reference value creation processing”, the reference value creating unit 14a stores the reference value information in the storage unit 15.

Note that, in the flowchart illustrated in FIG. 8, the reference value creating device 1a performs processing in the order of step ST21, step ST22, and step ST23, but the order of processing of steps ST21 to ST23 is not limited thereto. The reference value creating device 1a just needs to perform the processing of step ST23 before performing the processing of step ST24.

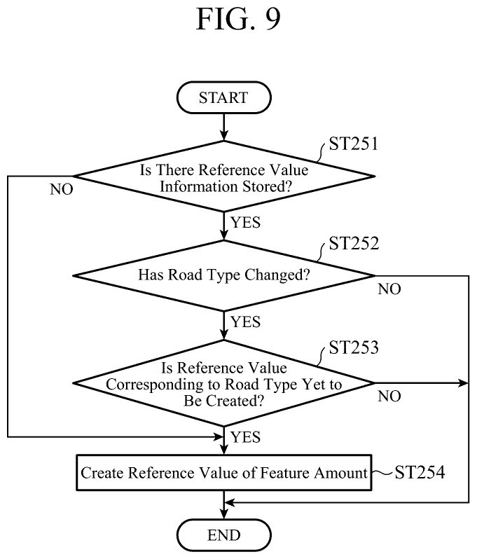

Further, in the flowchart illustrated in FIG. 8, the reference value creating device 1a performs the processing of step ST24 before step ST25, but this is merely an example. For example, when the selecting unit 16 selects the target feature amount in response to a determination result that the road type of the road on which the vehicle 10 is traveling has changed by the reference value creating unit 14a, the processing of step ST24 is performed during the reference value creation processing of step ST25 (after step ST253 in FIG. 9 described later).

FIG. 9 is a flowchart for explaining a specific operation of step ST25 in FIG. 8.

The specific operations in steps ST251 to ST253 in FIG. 9 are similar to the specific operations in steps ST41 to ST43 described in the first embodiment, respectively, and thus duplicate description will be omitted.

When it is determined in step ST253 that the reference value corresponding to the changed road type is yet to be created (when “YES” in step ST253), or when it is determined in step ST251 that the reference value information is not stored in the storage unit 15 (when “NO” in step ST251), the reference value creating unit 14a creates the reference value for each feature amount on the basis of the feature amount information acquired by the feature amount acquiring unit 13 in step ST23 of FIG. 8 (step ST254).

Here, when the road type determined by the road determining unit 12 has changed and it is determined that the reference value corresponding to the changed road type should be created, that is, it is determined in step ST253 that the reference value corresponding to the changed road type is yet to be created (“YES” in step ST253), the reference value creating unit 14a creates the reference value on the basis of the feature amount information output from the feature amount acquiring unit 13 for the target feature amount selected by the selecting unit 16 in step ST24 of FIG. 8 among the feature amounts, and sets the reference value to the same value as the reference value created immediately before for feature amounts other than the target feature amount.

Then, the reference value creating unit 14 stores the reference value information in the storage unit 15.

As described above, when the road type of the road on which the vehicle 10 is traveling has changed, the reference value creating device 1a can create the reference value again only for the feature amount in which an individual difference in the tendency of the change in the feature amount depending on the road type is likely to occur by dividing the feature amount into the feature amount (target feature amount) for creating the reference value again and the feature amount for using the reference value that has been used to estimate the alertness level of the occupant until then. As a result, the alertness level estimating device 2 can accurately estimate the alertness level of the occupant in consideration of the individual difference using the reference value created by the reference value creating device 1a.

As described above, the reference value used for estimating the alertness level of the occupant is preferably created immediately after the start of driving at which it is assumed to be in the awake state. However, since the road type affects the feature amount, it is necessary to review the reference value when the road type changes.

Therefore, in the first embodiment, when it is determined that the reference value corresponding to the changed road type should be created due to a change in the road type of the road on which the vehicle 10 is traveling, the reference value creating device 1 creates the reference values for all the feature amounts of the first feature amount, the second feature amount, the third feature amount, the fourth feature amount, and the fifth feature amount on the basis of the feature amount information.

Here, not all the feature amounts are feature amounts having a large change depending on the road type. In consideration of this point, when the road type of the road on which the vehicle 10 is traveling has changed, the reference value creating device 1a according to the second embodiment creates the reference value again only for the feature amount in which the individual difference in the tendency of the change in the feature amount depending on the road type is likely to occur. Specifically, in the reference value creating device 1a, when it is determined that the reference value corresponding to the changed road type should be created due to a change in the road type of the road on which the vehicle 10 is traveling, for example, the reference value creating unit 14a creates the reference value based on the feature amount information again for the target feature amount among the first feature amount, the second feature amount, the third feature amount, the fourth feature amount, and the fifth feature amount, and uses the reference value that has been used to estimate the alertness level of the occupant in the alertness level estimating device 2 so far for the reference value of the feature amount other than the target feature amount.

The reference value creating device 1a sets the reference value of the feature amount in which it is assumed that the individual difference in the tendency of the change in the feature amount depending on the road type is not likely to occur not as the reference value created from the feature amount obtained when the vehicle 10 travels on the road of the changed road type, but as the reference value created on the basis of the reference value used at the time of traveling on the road before the road type is changed, in other words, the feature amount at a time point closer to the start of driving than the current time (the time point at which the occupant is assumed to be closer to an awake state). Thus, the reference value creating device 1a can achieve more accurate estimation of the alertness level of the occupant by preventing a decrease in the alertness level of the occupant from being hardly detected while preventing the occurrence of erroneous detection of the alertness level of the occupant due to an increase in the difference between the reference value and the feature amount due to a change in the road type.

The hardware configuration of the reference value creating device 1a according to the second embodiment is similar to the hardware configuration of the reference value creating device 1 described with reference to FIGS. 6A and 6B in the first embodiment.

In the second embodiment, the functions of the vehicle-related information acquiring unit 11, the road determining unit 12, the feature amount acquiring unit 13, the reference value creating unit 14a, and the selecting unit 16 are implemented by the processing circuit 51. That is, the reference value creating device 1a includes the processing circuit 51 for creating the reference value of the feature amount used for estimating the alertness level of the occupant on the basis of the vehicle-related information and the feature amount information.

The processing circuit 51 executes the functions of the vehicle-related information acquiring unit 11, the road determining unit 12, the feature amount acquiring unit 13, the reference value creating unit 14a, and the selecting unit 16 by reading and executing the program stored in the memory 55. That is, the reference value creating device 1a includes the memory 55 for storing a program that results in execution of steps ST21 to ST25 of FIG. 8 described above when executed by the processing circuit 51. Further, it can also be said that the program stored in the memory 55 causes a computer to execute a procedure or a method performed by processing of the vehicle-related information acquiring unit 11, the road determining unit 12, the feature amount acquiring unit 13, the reference value creating unit 14a, and the selecting unit 16.

The reference value creating device 1a includes the input interface device 52 and the output interface device 53 that perform wired communication or wireless communication with a device such as the alertness level estimating device 2 or the vehicle-related information acquiring device 3.

Note that, in the second embodiment described above, the reference value information stored in the storage unit 15 may not be deleted. In this case, instead of the (first condition) as described above, for example, “the reference value information corresponding to the occupant is stored in the storage unit 15 (third condition)” is set as the reference value creation condition. In step ST251 of FIG. 9, the reference value creating unit 14a determines whether or not the reference value information corresponding to the occupant is stored in the storage unit 15.

In addition, in the second embodiment described above, a general reference value that is used only until the reference value creating unit 14a stores the reference value information is stored in the storage unit 15, and the alertness level estimating unit 25 may estimate the alertness level of the occupant using the general reference value. In the reference value creating device 1a, when causing the storage unit 15 to store the reference value information for the first time, the reference value creating unit 14a notifies the alertness level estimating unit 25 of the fact. Upon receiving the notification from the reference value creating unit 14a, the alertness level estimating unit 25 stops using the general reference value.

In addition, in the second embodiment described above, for example, the reference value creating device 1a may be mounted on the alertness level estimating device 2, or the alertness level estimating device 2 may be mounted on the reference value creating device 1a. In addition, in the above-described second embodiment, for example, the image acquiring unit 21, the state detecting unit 22, and the feature amount calculating unit 23 may be provided in the reference value creating device 1a, or may be provided in other places where the alertness level estimating device 2 and the reference value creating device 1a can refer to.

In addition, in the above-described second embodiment, some of the vehicle-related information acquiring unit 11, the road determining unit 12, the feature amount acquiring unit 13, the reference value creating unit 14a, the selecting unit 16, the image acquiring unit 21, the state detecting unit 22, the feature amount calculating unit 23, the road information acquiring unit 24, and the alertness level estimating unit 25 may be included in an in-vehicle device of a vehicle, and the others may be included in a server connected to the in-vehicle device via a network.

Further, in the second embodiment described above, the occupant whose alertness level is to be estimated is not limited to the driver, and includes an occupant in the passenger seat or an occupant in the rear seat. Furthermore, a plurality of occupants may be targets for estimating the alertness level. In this case, the reference value creating device 1a creates a reference value for each occupant and stores the reference value information for each occupant in the storage unit 15.

As described above, according to the second embodiment, the reference value creating device 1a includes the selecting unit 16 to select, when the road type of the road on which the vehicle 10 is traveling has changed, a target feature amount for which the reference value corresponding to the changed road type is to be created from among the plurality of types of the feature amounts, and when the road type determined by the road determining unit 12 has changed, the reference value creating unit 14a creates the reference value on the basis of the feature amount information regarding the feature amount calculated on the basis of the captured image captured in the vehicle 10 traveling on the road after the road type is changed for the target feature amount with respect to the reference value of the feature amount corresponding to the changed road type, and sets, as the reference value, the same value as the reference value corresponding to the road type of the road on which the vehicle 10 has been traveling before the change of the road type for the feature amount other than the target feature amount. Therefore, when the road type of the road on which the vehicle 10 is traveling changes, the reference value creating device 1a can review the reference value only for the feature amount in which the individual difference in the tendency of the change in the feature amount depending on the road type is likely to occur by dividing the feature amount into the feature amount (target feature amount) for creating the reference value again and the feature amount for using the reference value that has been used to estimate the alertness level of the occupant until then.

Note that, in the present disclosure, free combinations of the embodiments, modifications of any components of the embodiments, or omissions of any components in the embodiments are possible.

### INDUSTRIAL APPLICABILITY

A reference value creating device according to the present disclosure can create a reference value for estimating an alertness level of an occupant in consideration of an individual difference in a tendency of a change in a feature amount depending on a road type.

### REFERENCE SIGNS LIST

1, 1a: reference value creating device, 11: vehicle-related information acquiring unit, 12: road determining unit, 13: feature amount acquiring unit, 14, 14a: reference value creating unit, 15: storage unit, 16: selecting unit, 2: alertness level estimating device, 21: image acquiring unit, 22: state detecting unit, 23: feature amount calculating unit, 24: road information acquiring unit, 25: alertness level estimating unit, 3: vehicle-related information acquiring device, 4: imaging device, 10: vehicle, 51: processing circuit, 52: input interface device, 53: output interface device, 54: processor, 55: memory

1. A reference value creating device, comprising:
processing circuitry configured to
acquire vehicle-related information related to a vehicle;
determine a road type of a road on which the vehicle is traveling on a basis of the acquired vehicle-related information;
acquire feature amount information regarding a feature amount of an occupant of the vehicle calculated on a basis of a captured image including a range in which at least a face of the occupant of the vehicle is present; and
create a reference value of the feature amount for estimating an alertness level of the occupant on a basis of the acquired feature amount information, and create, when the determined road type has changed, the reference value of the feature amount corresponding to the changed road type on a basis of the feature amount information regarding the feature amount calculated on a basis of the captured image captured in the vehicle traveling on the road of the changed road type.

2. The reference value creating device according to claim 1, wherein
the processing circuitry creates the reference value of the feature amount on a basis of the feature amount information acquired in a reference value creation target period or the feature amount information corresponding to the captured image for a number of frames for reference value creation.

3. The reference value creating device according to claim 1, wherein
the feature amount includes a plurality of feature amounts, each of which includes a different road type,
the processing circuitry is configured to select, when the road type of the road on which the vehicle is traveling has changed, a target feature amount for which the reference value corresponding to the changed road type is to be created from among the plurality of feature amounts, and
when the determined road type has changed, the processing circuitry creates the reference value on a basis of the feature amount information regarding the feature amount calculated on a basis of the captured image captured in the vehicle traveling on the road after the road type is changed for the target feature amount, with respect to the reference value of the feature amount corresponding to the changed road type, and sets, as the reference value, a same value as the reference value corresponding to the road type of the road on which the vehicle has been traveling before the change of the road type for the feature amount other than the target feature amount.

4. The reference value creating device according to claim 3, wherein
the processing circuitry is configured to acquire information regarding the alertness level of the occupant estimated on a basis of the reference value of the feature amount and the feature amount in a set period before the road type changes, and selects the feature amount assumed to be likely to change depending on the road type as the target feature amount when the alertness level of the occupant decreases.

5. The reference value creating device according to claim 1, wherein
each road type includes an ordinary road, an automobile exclusive road, or an expressway.

6. An alertness level estimating device, comprising:
second processing circuitry configured to
acquire road information regarding the road type determined by the reference value creating device according to claim 1; and
estimate the alertness level of the occupant on a basis of the reference value of the feature amount corresponding to the road type indicated by the acquired road information and the feature amount, the reference value being created by the reference value creating device.

7. The alertness level estimating device according to claim 6, wherein
the second processing circuitry is configured to estimates the alertness level of the occupant on a basis of a difference between the feature amount and the reference value of the feature amount and a machine learning model that uses the difference between the feature amount and the reference value of the feature amount as an input and outputs information related to the alertness level.

8. The alertness level estimating device according to claim 6, wherein
the second processing circuitry configured to estimate the alertness level of the occupant on a basis of the difference between the feature amount and the reference value of the feature amount and a set alertness level estimation condition.

9. A reference value creating method, comprising:
acquiring vehicle-related information related to a vehicle;
determining a road type of a road on which the vehicle is traveling on a basis of the acquired vehicle-related information;
acquiring feature amount information regarding a feature amount of an occupant of the vehicle calculated on a basis of a captured image including a range in which at least a face of the occupant is present; and
creating a reference value of the feature amount for estimating an alertness level of the occupant on a basis of the acquired feature amount information, and creating, when the determined road type has changed, the reference value of the feature amount corresponding to the changed road type on a basis of the feature amount information regarding the feature amount calculated on a basis of the captured image captured in the vehicle traveling on the road of the changed road type.
